(19) United States
(12) Patent Application Publication
(10) Pub. No.: US 2003/0083680 A1
(43) Pub. Date: May 1, 2003

(54) Tongue cleaner

(76) Inventor: Pierre J. Jousson, Geneva (CH)

Correspondence Address: Pitney, Hardin, Kipp and Szuch, 711 Third Avenue, New York, NY, 10017-4014, US

(21) Appl. No.: 09/999,059

(22) Filed: Oct. 31, 2001

(51) Int. Cl. A61B 17/24

(52) U.S. Cl. 606/161

(57) ABSTRACT

A tongue cleaner having a housing in which a mechanical scraping blade is attached to a spraying fixture which can draw from a water source possibly with a chemical additive. When the user applies scraping blade of the tongue cleaner to the tongue, the surface of the tongue and the papillac of the tongue are exposed and sprayed with water; engaging and reducing bacteria buildup, which is the cause of Halitosis or bad breath.

### BACKGROUND OF THE INVENTION

1. Field of the Invention

This invention relates to a device for cleaning the tongue of the user wherein the device combines mechanical scraping of the tongue with the spray action of a liquid, preferably water and possibly water with a chemical additive. When operated, this tongue cleaning device should reduce the amount of odorous bacteria located at the surface and in the papillac of the tongue.

2. Brief Description of the Prior Art

In the prior art, it is well known that halitosis or bad breathe is caused by a buildup of bacteria in the mouth where there is an emission of hydrogen sulfide and sulfide methlmercaptan from these bacteria. These bacteria are needed because they assist the digestion process. However, for some persons, these particular bacteria are found in higher numbers causing noticeable cases of halitosis or bad breath. Instances of these noticeable cases of bad breath are evenly split between men and women. The bacteria of the condition known as Halitosis, are classified as anaerobics meaning that these bacteria grow in an area where oxygen is not present. For the tongue, this area is not exclusively on the surface of the tongue but is mostly between the papillac that make up the body of the human tongue.

Various devices exist that attempt the elimination of the bacteria which causes Halitosis. In U.S. Pat., Des. 267,508 a tongue cleaning device is disclosed with a depiction of tongue scraper. The tongue scraper has an edge which is run over the surface of the tongue in a scraping motion. This scraping eliminates a minimal amount of bacteria since most bacteria grow beneath the surface in the papillac of the tongue. Since scraping the tongue alone is inadequate, continued scraping for effect without noticeable results could lead to soreness or even bleeding on the tongue.

Other methods for treating Halitosis include brushing the teeth and using dental floss on the teeth; the use of antibiotics; the application of oral rinses to the tongue; and treatments which convert the bacteria into non-odorous forms. Brushing teeth and using dental floss on the teeth are usually inadequate methods to rid the mouth of such bacteria, because the build-up is located on the tongue and not the teeth. Antibiotics have only a temporary effect of eliminating all bacteria but since the bacteria are beneficial, the body will produce more bacteria of possibly resistant strains to replace the eliminated bacteria. The resistant strains could eventually be impervious to the continued use of antibiotics and the problem of over-produced bacteria will return. Also, continued use of antibiotics may lead to infections, in which the infections result from an imbalance of normal oral bacterial levels. Oral rinses which pass the tongue, effect the surface of the tongue and only incidentally reach the papillac of the tongue if at all. Treatments which convert bacteria into non-odorous forms can be lengthy and may require clinical observance which can lead to expense.

It is seen from the foregoing that there is a need for a device which overcomes the disadvantages found in the prior art concerning the reduction of buildup of odorous bacteria on the surface and in the body of the tongue. Such as device should be able to reduce in an effective and less costly manner; the amount of bacteria buildup on the surface of the tongue and in between the papillac which make up the body of the tongue.

### SUMMARY OF THE INVENTION

It is therefore an object of the present invention to provide a device which reliably removes bacteria buildup in between the papillac of the tongue.

It is therefore a further object of the present invention to provide a device which reliably removes bacteria buildup on the surface of the tongue.

It is therefore a still further object of the present invention to provide a device for removing bacteria buildup on the surface of the tongue and in the papillac of the tongue in which the device can be disassembled for cleaning.

It is therefore a still further object of the present invention to provide a device for removing bacteria buildup on the surface of the tongue and in the papillac of the tongue in which the device can be disassembled for changing worn out or broken components.

It is therefore a still further object of the present invention to provide a device which is portable in size.

To attain the objects described, there is provided a device which is a mechanical scraper having a sprayer with a nozzle. This sprayer can draw on a water source that has effective chemical additives such as chlorine dioxide. The device achieves the best results when attached with an oral irrigator that can provide pressurized liquid for high energy spraying. High energy spraying would enhance the ability to reach the papillac of the tongue.

### BRIEF DESCRIPTION OF THE DRAWINGS

Thus by the present invention its objects and advantages will be realized, the description of which should be taken in conjunction with the drawings wherein:

FIG. 1 is a bottom cross-sectional view of the tongue cleaner.

FIG. 2 is a side cross-sectional view of the tongue cleaner.

FIG. 3 is a cross-sectional view of the mouthpiece of the tongue cleaner, looking from the housing of the tongue cleaner.

FIG. 4 is an end view of the mouthpiece of the tongue cleaner.

### DETAILED DESCRIPTION OF THE PREFERRED EMBODIMENTS

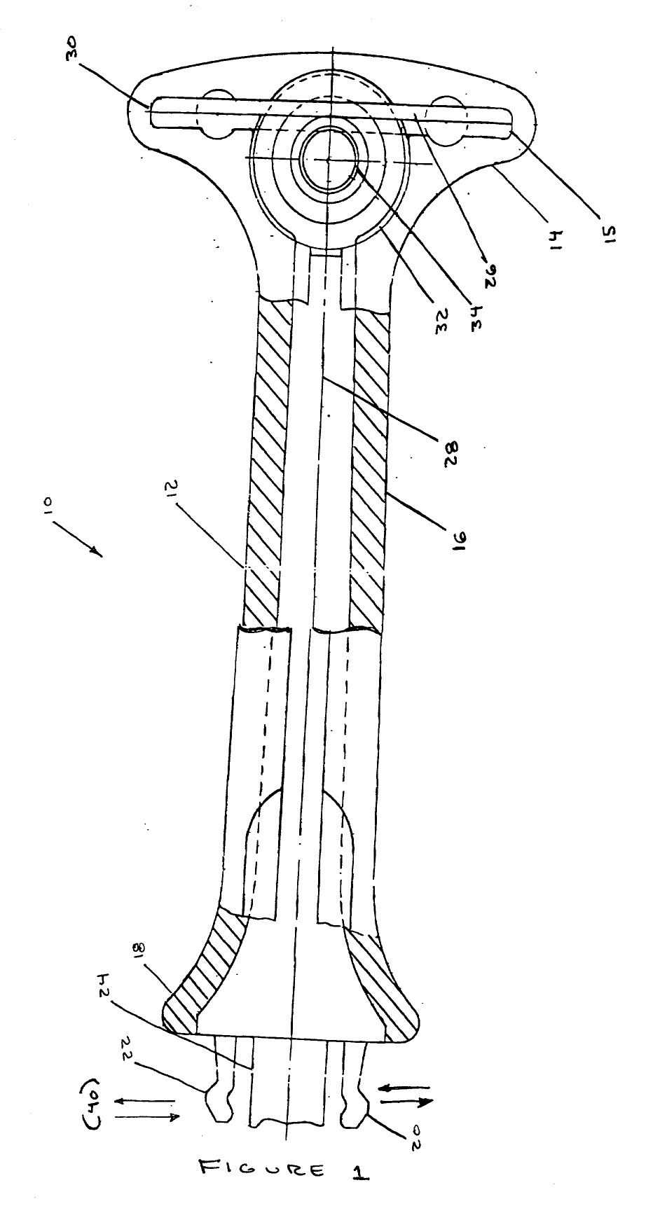

Referring now to the drawings wherein like numerals refer to like elements throughout the several views, one sees that FIG. 1 is a bottom cross-sectional view of the tongue cleaner 10 of the present invention. The tongue cleaner 10 is generally a housing 12 shaped to be grasped by the user to allow the user to grip and manipulate during tongue cleaning. The shape of housing 12 is an elongated rounded body with varying circumferences.

An mouthpiece 14 is located on the distal end and integral with housing 12. From the mouthpiece 14, the circumference of the housing 12 decreases to a gripping area 16, which is a majority of the outside surface area of the housing 12. From the gripping area 16, the housing 12 increases to a slightly larger circumference at the liquid source attachment section 18. The enlarged circumference of liquid source attachment section 18 allows an oral irrigator to conformably fit to the tongue cleaner 10. At the liquid source attachment section 18, two mating prongs 20,22 and a liquid source extension 24 are also provided for the fit of an oral irrigator.

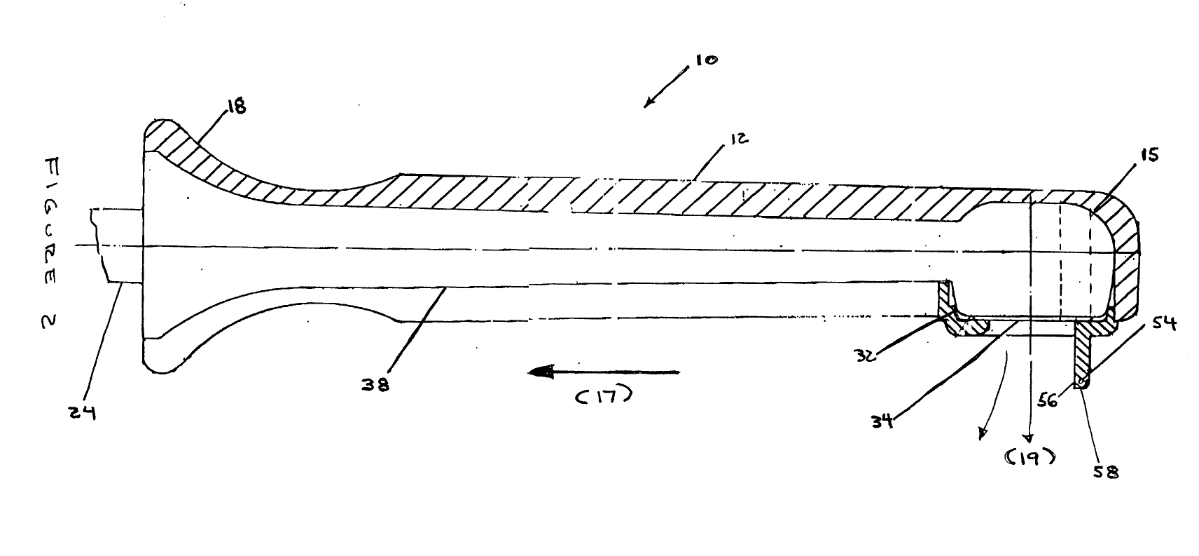

When the tongue cleaner 10 is being used, the housing 12 is pulled in a lateral motion on the surface of the tongue of the user as indicated by direction arrow 17 in FIG. 2. Upon simultaneous activation of the oral irrigator, pressurized liquid emits from a spray nozzle as indicated by spray pattern 19. These two concurrent actions expose the surface and the papillac of the tongue to the cleansing action of the pressurized liquid thereby reducing the amount of anaerobic bacteria residing in these areas.

In FIG. 1, mouthpiece 14 is an semi-elliptical housing whose longitudinal axis 26 is perpendicular to the longitudinal axis 28 of the housing 12. The mouthpiece 14 is rounded at its boundaries for comfortable movement within the mouth of the user. The width of mouthpiece 14 is sized for fitting within the mouth of the user. The mouthpiece 12 also allows detachment of the scraper section 15 for cleaning and replacement of parts. Recess 30 located on the underside of mouthpiece 14 and along longitudinal axis 26, is provided to allow insertion of scraper section 15. Located near recess 30 and towards gripping area 16 is spray port 32.

Integral to spray port 32 is spray nozzle 34. In FIG. 2, spray nozzle 34 is sized to produce microfractionated jets of pressurized liquid with a pulsation frequency between 1500 and 6000 pulsations per minute and a spraying surface of 0.5-4 square millimeters when the internal integrated pressure is between 1 and 6 bars. The spray pattern 19 of microfractionated jets would emit from spray nozzle 34. Pressurized liquid, needed for the spray pattern 19, may be water with or without chemical additive supplied through liquid channel 38. Liquid channel 38 is a hollowed section interior to housing 12 and following the longitudinal axis 28 of housing 12. Pressured water to liquid channel 38 is supplied by liquid source extension 24, when liquid source attachment section 18 is coupled with an external liquid source.

Liquid source attachment section 18 preferably couples with a “Broxo-Jet” or “Broxototal” oral irrigator manufactured by Les Produits Associates of Geneva Switzerland, herein incorporated by reference. Alternative liquid sources suitable for the purpose of providing pressurized liquid to the present invention may be used. When provided with the “Broxo-Jet” or “Broxototal” oral irrigators, spray nozzle 34 produces micro-fractioned jets with a pulsated frequency between 1500 and 5000 pulsations/minute, an internal integrated pressure between 1 and 6 bars and spraying surface between 0.5 and 4 square millimeters.

In FIG. 1, prongs 20, 22 are integral with and extrude from liquid attachment section 18 as flexible lengths for attachment to an oral irrigator. The flexibility of prongs 20, 22 allow them to be squeezed to fit into corresponding apertures on an oral irrigator and when placed prongs 20, 22 snap back to their original position securing the oral irrigator. The direction of flexibility of prongs 20, 22 is indicated by arrows 40. Liquid source extension 24 is a hollowed cylinder used as an additional attachment section to fit with the liquid orifice of an oral irrigator.

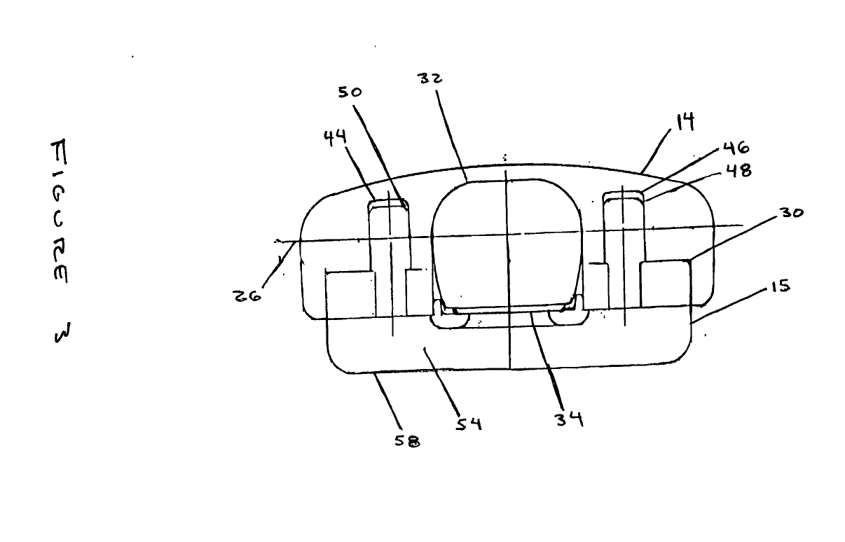

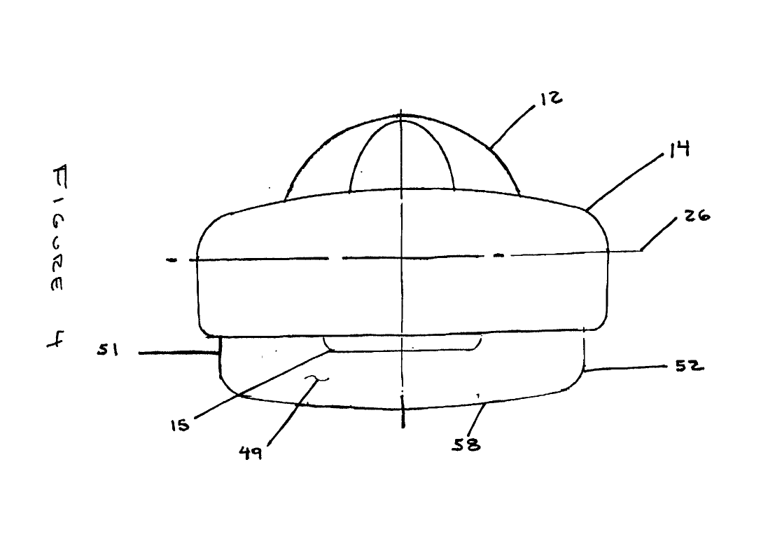

In FIG. 3 the attachment of scraper section 15 is shown, where the figure depicts a cross-sectional end view of mouthpiece 14 of tongue cleaner 10, looking away from housing 12. Mouthpiece 14 has recessed areas 44, 46 sized to accommodate attachment fixtures 48, 50 of scraper section 15. In FIG. 4 the shape of scraper 49 is depicted which is an end view of mouthpiece 14 looking towards housing 12 with scraper 49 of scraper section 15 depicted. Scraper 49 is a thickened blade having sides edges 51, 52; opposite faces 54, (56 in FIG. 2); and an edge 58. Scraper 49 has a width of twenty millimeters plus or minus 50% in the direction of axis 26. Edge 58 and side edges 51, 52 which are in contact with the tongue of the user are flat or have radius not smaller than 50 millimeters. Scraper 49 is preferably made of plated gold however alternatives suitable for the purpose of the present invention may be used.

Thus by the present invention its objects and advantages are realized and although preferred embodiments have been disclosed and described in detail herein, its scope should be determined by that of the appended claims.

We claim:

1. A tongue cleaning device operable with pressurized liquid and the actions of a user, said tongue cleaning device comprising:
a housing, wherein said housing provides a conduit for pressurized liquid,
an mouthpiece integral to the distal end of said housing wherein said mouthpiece provides a passage for said pressurized liquid from said housing;
a scraper section coupled to said mouthpiece wherein said scraper section engages the surface of the tongue of the user in a scraping operation;
a spray nozzle coupled to said mouthpiece wherein said spray nozzle administers pressurized liquid provided by said mouthpiece to the surface and papillac of the tongue;
a liquid attachment section coupled to the proximal end of said housing wherein said liquid attachment section can be mechanically coupled to a pressurized liquid source.

2. The tongue cleaning device of claim 1, wherein said scraper section is mechanically detachable from said mouthpiece.

3. The tongue cleaning device of claim 1, wherein said spray nozzle emits microfractionated jets of pressurized liquid.

4. The tongue cleaning device of claim 3 wherein said microfractionated jets have a pulsation frequency between 1500 and 6000 pulsations per minute.

5. The tongue cleaning device of claim 1 wherein said spray nozzle emits a spray pattern that covers a spray surface of 0.5-4 square millimeters at a pressure from 1 to 6 bars.

6. A method of reducing bacteria buildup on the surface and in the body of the tongue comprising the steps of:
providing the tongue cleaning device of claim 1;
placing said device on the surface of the tongue wherein the scraper section is in contact with the back of the tongue and wherein the longitudinal axis of the device is aligned and parallel with the longitudinal axis of the tongue of the user;
applying pressurized liquid to said tongue cleaner, wherein a spray pattern is emitted from the spray nozzle of the device;
applying light downward force in the direction of the scraper wherein the scraper indents the surface of the tongue;
moving the device in a scraping motion with a lateral direction towards the entrance of mouth;
maintaining light downward pressure to the surface of the tongue thereby exposing the surface and body of the tongue to the pressurized liquid; and
repeating said scraping motion to all areas of the tongue thereby reducing bacteria build up on the surface and body of the tongue.

7. The method of claim 6, wherein the method is used to clean the surface and body of the tongue.
